(19) United States
(12) Patent Application Publication
(10) Pub. No.: US 2023/0218941 A1
(43) Pub. Date: Jul. 13, 2023

(54) PLAY EXPERIENCE SYSTEM AND PHYSICAL DEVELOPMENT STATUS ACQUISITION METHOD USING SAME

(71) Applicant: KOREA ELECTRONICS TECHNOLOGY INSTITUTE, Seongnam-si, Gyeonggi-do (KR)

(72) Inventors: Yeonkug MOON, Seoul (KR); Donghyun LEE, Seoul (KR); Minjoon KIM, Seongnam-si, Gyeonggi-do (KR); Sunghun CHAE, Seongnam-si, Gyeonggi-do (KR); Youngbo SHIM, Seoul (KR)

(21) Appl. No.: 17/784,164

(22) PCT Filed: Dec. 11, 2019
(86) PCT No.: PCT/KR2019/017478

§ 371 Date: Jun. 10, 2022

(30) Foreign Application Priority Data
Dec. 10, 2019 (KR) 10-2019-0164162

(51) Int. Cl. A63B 17/00; A63B 24/00

(52) U.S. Cl. CPC A63B 17/00; A63B 24/0062; A63B 2024/0068; A63B 2230/06; A63B 2220/833; A63B 2230/20; A63B 2220/803; A63B 69/0048

(57) ABSTRACT

The disclosure of the present invention relates to a play experience system and a physical development status acquisition method using the same, which can precisely measure physical development items of users including children and juveniles by using play-type experience devices, and can acquire experience data based physical development status by analyzing a correlation between an experience instrument itself and the physical development item.

### CROSS-REFERENCE TO RELATED APPLICATION

The present application is a national stage filing under 35 U.S.C § 371 of PCT application number PCT/KR2019/017478 filed on Dec. 11, 2019, which is based upon and claims the benefit of priorities to Korean Patent Application No. 10-2019-0164162, filed on Dec. 10, 2019, in the Korean Intellectual Property Office, which are incorporated herein in their entireties by reference

### TECHNICAL FIELD

The disclosure relates to a play experience system and a physical development status acquisition method using the same.

More specifically, the disclosure relates to a play experience system and a physical development status acquisition method using the same, which can precisely measure physical development items of users including children and juveniles by using play-type experience devices, and can acquire experience data based physical development status by analyzing a correlation between an experience instrument itself and the physical development item.

### BACKGROUND ART

In general, a person can stay healthy by being physically active along with mental activity. In the past, almost everyone moved their bodies in their everyday lives, but in recent times, many people need to exercise separately because of an increase in their indoor activities.

Therefore, in places nationwide that have good scenery, trails or walking areas have been established to provide conditions for various exercises to be undertaken in a pleasant environments.

However, because people living in a city need to make many preparations in order to do exercise on trails or promenades, and the conditions are often unfavorable, it is not easy for people living in a city to use trails or promenades.

In addition, users that have less time for exercise mostly use gyms. If it is not feasible, they often prepare fitness equipment at home for exercise. For this, various kinds of fitness equipment with excellent performance are available.

However, in the case of exercising alone in a gym or at home, a problem is that exercise is not undertaken continuously because the user loses interest and easily becomes tired of the exercise.

Especially in the case of children, because amusement facilities for children such as play areas and jungle gyms are merely physical amusement facilities only for simple play and having fun and are composed of only facilities and equipment with a lack of professionalism and limited usage, there is also a problem in that the frequency of return visits decreases. Also, children's understanding of or accessibility to high-tech culture is degraded.

As a scheme for solving the above problems in the related art, domestic patent application publication no. 10-2019-0098870 discloses a Medi-Sportainment physical strength measurement and analysis system for play-type child growth and development, which measures physical strength, including such as body, balance, and sensation while a user has fun.

However, according to the related art document, because only one basic physical strength measurement is taken of physical strength items evaluated through physical strength measurement equipment per evaluation items provided in an experience room, there is a problem in that it is not possible to obtain information on results showing children's precise basic physical strength.

In addition, in the case that all children do not use the physical strength items evaluated by the physical strength measurement equipment per evaluation item provided in the space of the experience room, accurate measurement of the basic physical strength items is not possible; thus, there is a problem in that it is not possible to use result information showing the basic physical strength items measured as above as standard information for basic physical strength items.

Further, since a physical development status acquisition method in the related art is merely a method for acquiring one physical development status through one experience instrument, that is, a simple action-oriented limited method for measuring a single physical development item, and does not consider related variables for other physical development items excluding the physical development item intended to be measured, there is a problem in that it is groundless whether measurement data measured by the measurement instrument is accurately related to the physical development item to be acquired.

Accordingly, there is a need for a new scheme for solving the above problems in the related art.

(Patent Document 1) Korean Patent Application Publication No. 10-2019-0098870 (Publication date: Aug. 23, 2019)

### DISCLOSURE OF INVENTION

### Technical Problem

The disclosure is proposed to solve the above problems in the related art, and an aspect of the disclosure is to provide a play experience system and a physical development status acquisition method using the same, in which a plurality of experience rooms constituting an experience device for play-type experiences are configured as a plurality of variable modules disposed in the form of courses. Also, by variably configuring weight values for the user's physical development items to be measured in the plurality of experience rooms, a user's physical development items can be measured comprehensively and precisely, and standard information on the physical development items can be updated more accurately and precisely through a continuous feedback of information (data) values of the measured physical development items into the existing information (data) values.

Another aspect of the disclosure is to provide a play experience system and a physical development status acquisition method using the same, which can accurately acquire all pieces of desired information on a plurality of physical development items by accurately analyzing the correlation between measurement information obtained from an experience through an experience instrument and each of the plurality of physical development items.

### Solution to Problem

In order to achieve the aspects as described above, a play experience system according to an embodiment of the disclosure may include an experience device including a plurality of experience rooms configured to provide spaces in which a user can carry out a play-type experience and composed of variable modules that are connected to form a course, and a plurality of experience instruments provided in each of the plurality of experience rooms for the user to experience the play-type experience so that physical development items of the user are measured comprehensively; a sensor device including at least one or more sensors being disposed in a predetermined area of each of the plurality of experience instruments and configured to measure comprehensively the physical development items while the user experiences the plurality of experience instruments; and a processing device configured to receive body composition information and heart rate information of the user and comprehensive measurement information on the physical development items of the user from the at least one or more sensors, and to obtain result information on a current level of the physical development items of the user by comparing the received comprehensive measurement information on the physical development items with pre-stored standard information on the physical development items.

A physical development status acquisition method using a play experience system according to an embodiment of the disclosure may include steps of: receiving, from a sensor device wherein an experience device includes a plurality of experience instruments composed of variable modules and the sensor device, measurement information per each of the experience instruments corresponding to the experience instruments, respectively; deriving a first correlation matrix composed of first correlation coefficients about a correlation among the measurement information per each of the experience instruments; determining a number of a principal component in which each eigenvalue of components constituting the first correlation matrix is larger than a predetermined eigenvalue; deriving a second correlation matrix capable of converting the measurement information per each of the experience instruments into information on a plurality of physical development items in accordance with the number of the principal component; and converting the measurement information per each of the experience instruments into the information on the plurality of physical development items by using the second correlation matrix.

### Advantageous Effects of Invention

The play experience system and the physical development status acquisition method using the same according to the embodiment of the disclosure can achieve the following effects.

1. Because the experience device for the play-type experience is configured as variable modules disposed in the form of courses, the physical development items of the user can be measured comprehensively and precisely.

2. Because the result information on the user's physical development items measured using the plurality of experience instruments is continually updated, the result information can be used as the physical development standard information.

3. Because the plurality of experience rooms are configured as variable modules, the level of difficulty of the plurality of experience instruments being provided respectively in the plurality of experience rooms can be adjusted; thus a user having repeated experiences cannot lose interest or stop having fun.

4. All pieces of desired information on the plurality of physical development items can be accurately acquired by accurately analyzing the correlation between the measurement information obtained from the experience through the experience instrument and each of the plurality of physical development items.

### BRIEF DESCRIPTION OF DRAWINGS

FIG. 1 is a configuration diagram schematically illustrating a play experience system according to an embodiment of the disclosure.

FIG. 2 is a diagram explaining the structure of a first experience instrument according to an embodiment of the disclosure.

FIG. 3 illustrates a state in which a star is awarded for physical development items measured comprehensively through the first experience instrument of FIG. 2.

FIG. 4 is a diagram explaining the structure of a second experience instrument according to an embodiment of the disclosure.

FIG. 5 illustrates a state in which a star is awarded for physical development items measured comprehensively through the second experience instrument of FIG. 4.

FIG. 6 is a diagram explaining the structure of a third experience instrument according to an embodiment of the disclosure.

FIG. 7 illustrates a state in which star is awarded for physical development items measured comprehensively through the third experience instrument of FIG. 6.

FIGS. 8A and 8B are flowcharts of a physical development status acquisition method using a play experience system according to an embodiment of the disclosure.

FIG. 9 is a perspective view of a play experience system used for a physical development status acquisition method using the play experience system according to an embodiment of the disclosure.

FIG. 10 is a configuration diagram schematically illustrating a play experience system used for a physical development status acquisition method using the play experience system according to an embodiment of the disclosure.

FIG. 11 is a diagram illustrating a table representing an average, standard deviation, and the number of data for each of first to sixth experience instruments of a physical development status acquisition method using a play experience system according to an embodiment of the disclosure.

FIG. 12 is a diagram illustrating a first correlation matrix for first to sixth experience instruments of a physical development status acquisition method using a play experience system according to an embodiment of the disclosure.

FIG. 13 is a diagram illustrating a table representing eigenvalues by components configuring a first correlation matrix of a physical development status acquisition method using a play experience system according to an embodiment of the disclosure.

FIG. 14 is a diagram illustrating eigenvalues by components illustrated in FIG. 13 as a graph.

FIG. 15 is a diagram illustrating a table representing correlation values for principal components of first to sixth experience instruments of a physical development status acquisition method using a play experience system according to an embodiment of the disclosure.

FIG. 16 is a diagram illustrating correlation values for principal components of first to sixth experience instruments illustrated in FIG. 15 as a graph.

### MODE FOR THE INVENTION

Hereinafter, preferred embodiments of the disclosure will be described in detail with reference to the accompanying drawings. In the accompanying drawings, it is to be noted that the same drawing reference numerals are used for the same elements. Further, detailed explanation of related well-known functions or configurations incorporated herein will be omitted in the case where it is determined that they obscure the subject matter of the disclosure.

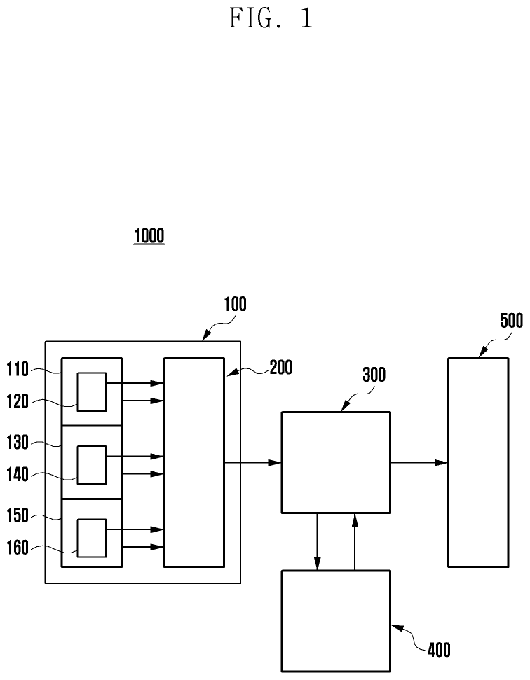

Hereinafter, a play experience system according to an embodiment of the disclosure will be described with reference to the accompanying drawings of FIGS. 1 to 7.

FIG. 1 is a configuration diagram illustrating schematically a play experience system according to an embodiment of the disclosure.

With reference to FIG. 1, a play experience system 1000 according to an embodiment of the disclosure includes a plurality of experience devices 100, a sensor device 200, and a processing device 300.

The experience device 100 according to an embodiment of the disclosure may include a plurality of experience rooms 110, 130, and 150 and a plurality of experience instruments 120, 140, and 160 provided in the plurality of experience rooms 110, 130, and 150, respectively.

The plurality of experience rooms 110, 130, and 150 may be provided with spaces in which a user can carry out a play-type experience, and they may be composed of variable modules so as to be connected in the form of courses.

More specifically, in configuring the play experience system, the plurality of experience rooms 110, 130, and 150 may be formed as a variable module structure so as to be disposed in various forms in accordance with the size of the space and the environmental conditions.

Each of the plurality of experience instruments 120, 140, and 160 according to an embodiment of the disclosure may be provided in the space of each of the plurality of experience rooms 110, 130, and 150 so that the user can have a play-type experience for comprehensive measurement of physical development items. Further, although the plurality of experience instruments 120, 140, and 160 may be connected in the form of courses, it is to be noted that the disclosure is not limited thereto.

In this case, each of the plurality of experience instruments 120, 140, and 160 may be implemented so that the user can undertake activities to cause the user to be interested and to have fun, and they may enable the user to measure comprehensively feature values of physical development items including muscular strength, muscular endurance, agility, quick reaction, flexibility, balance, and cardiorespiratory endurance.

Weight values may be given to some or all of the physical development items being measured in each of the plurality of experience instruments 120, 140, and 160, and the disposition of the plurality of experience instruments 120, 140, and 160 in the experience device may be provided variably in accordance with the weight values. Based on the user's physical development measurement items measured by each of the plurality of experience instruments 120, 140, and 160, result information on the physical development items at the current level of the user may be obtained.

Hereinafter, several examples of the plurality of experience instruments 120, 140, and 160 will be described in detail.

Meanwhile, although not illustrated, before the user enters into the first experience room 110 among the plurality of experience rooms 110, 130, and 150, the processing device 300 to be described later is provided with and stores basic personal information of the user, and the processing device 300 issues a personal membership number associated with the basic personal information. Thereafter, the user wears a wearable device (e.g., wrist band) with a recorded membership number to provide the user's bio-information, and a helmet on which a tag for safety protection and the user's location recognition is mounted. Here, the wearable device on which the membership number has been recorded may be provided integrally with the helmet.

In this case, the basic information may include a user's name, age, gender, and guardian information, and the bio-information may include information on the user's heart rate, temperature, and blood pressure. The basic information and the bio-information may be stored and managed in the processing device 300 to be described later by using network communication. Further, the user's body information, such as height and weight, may be measured through body information measurement equipment disposed on the side of a pre-experience instrument to be described later, and then such information may be stored in the processing device 300.

The sensor device 200 according to an embodiment of the disclosure may be disposed in any one area or location of each of the plurality of experience instruments 120, 140, and 160 provided in each of the plurality of experience rooms 110, 130, and 150, and it may include at least one or more sensors (not illustrated) transmitting the physical development measurement information measured while the user experiences the plurality of experience instruments 120, 140, and 160 to the processing device 300 to be described later.

As for the sensor device 200, at least one of a pressure sensor, a short-range sensor, a touch sensor, and a motion recognition sensor for measuring the physical development with respect to the user's muscular strength, muscular endurance, agility, quick reaction, flexibility, balance, and cardiorespiratory endurance is installed in each of the experience rooms and on each of the experience instruments, and it provides comprehensive measurement of the physical development items of the user while the user experiences the experience instruments 120, 140, and 160 while moving through the respective experience rooms 110, 130, and 150.

The processing device 300 according to an embodiment of the disclosure may receive the pre-measured body composition information and the heart rate information of the user and the measurement information on the physical development items of the user from the at least one sensor, and may obtain the result information on the current level of the physical development items of the user by comparing the received measurement information on the physical development items with the standard information on the pre-stored physical development items. The processing device 300 may be implemented by, for example, a server, a PC, and a microcontroller, but it is to be noted that the disclosure is not limited thereto.

Meanwhile, the processing device 300 may derive the correlation between the result information and the physical development items by using the information measured by the plurality of experience instruments 120, 140, and 160, and the plurality of experience instruments 120, 140, and 160 provide the weight values be given to some or all of the expected physical development items through the measured information.

The processing device 300 may calculate the result information on the current level of the physical development items of the user in contrast with the standard information on the pre-stored physical development items based on the measurement information on the physical development items of the user, being measured through the at least one sensor provided in each of the plurality of experience instruments 120, 140, and 160, during the user's entry of the first experience room 110 to the user's exit of the first experience room 110 and the pre-measured and stored heart rate change information.

Meanwhile, the standard information on the physical development items pre-stored in the processing device 300 may be obtained using a generally well-known physical activity promotion system (PAPS), and this will be explained simply as follows.

The physical development standard information for the body composition of the user may be obtained by using an in-body test. In this case, by using the in-body test, the user's weight, body mass index, body fat mass, and body fat percentage may be obtained. Further, the physical development standard information on the user's muscular strength may be obtained using a grip force meter, and for example, it may be obtained by measuring the maximum value of the grip force meter for a dominant one of both hands of the user. Further, the physical development standard information on the user's muscular endurance may be obtained by performing upper body roll up, and for example, it may be obtained by measuring the number of successes until the point of failure by an upper body roll up being performed by one cycle every 3 seconds. Further, the physical development standard information on the user's cardiorespiratory endurance may be obtained by performing a 15 m long round trip running or by measuring the heart rate through a step test. Further, the physical development standard information on the user's flexibility may be obtained through forward bending of the upper body in a seated state. Further, the physical development standard information on the quick reaction of the user may be obtained through a standing long jump. Further, the physical development standard information on the agility of the user may be obtained through a total physical response test or a side-step test. Lastly, the physical development standard information on the balance of the user may be obtained through the user standing on one leg with eyes closed.

Meanwhile, although not illustrated, separately from the experience device, the standard information on the physical development items, of which the correlation with some or all of the physical development items is verified, may be pre-obtained and used by using the information measured through a plurality of pre-experience instruments (not illustrated) being provided so as to measure individually the physical development items before the user has experience through the experience device.

The pre-experience instrument may be provided separately from the experience device 100, and it may be implemented as a structure similar to the experience device 100.

That is, the measurement information on the physical development items measured using the pre-experience instrument may be collected with respect to a plurality of users (e.g., 100 users having the same age), and information obtained by calculating an average value may be used as the standard information on the physical development items.

The standard information on the physical development items measured and obtained in the above method is stored in the processing device 300, and the processing device 300 collects the measurement information on the physical development items obtained by the user through the actual experience instruments 120, 140, and 160 from at least one sensor, and obtains the result information on the physical development items at the current level of the user by comparing the collected measurement information on the physical development items with the pre-stored standard information on the physical development items.

Meanwhile, in the case that a user having performed the measurement of the physical development items measures the physical development items again through the experience device 100 composed of the same experience instruments 120, 140, and 160, the processing device 300 according to an embodiment of the disclosure may adjust the experience difficulty for the experience device 100 based on the pre-stored physical development result information of the user. As an example of the difficulty adjustment of the experience device 100, the difficulty of the experience device 100 may be adjusted not only by controlling adjustment of the passage time of each of the plurality of experience rooms 110, 130, and 150 or adjustment of the time or frequency of button touch of the touch sensor, but also it may be adjusted by changing game (play) content being provided in the plurality of experience rooms 110, 130, and 150.

That is, the experience device 100 according to an embodiment of the disclosure may be implemented to be able to adjust the difficulty, and the user may not lose interest or stop having fun even if the user performs an activity repeatedly with the same experience device 100. Further, degradation of the measurement reliability, which may occur by the predicted behavior through the user's repeated experience with respect to the same configuration of the experience device 100, may be prevented.

As described above, the processing device 300 of the play experience system 1000 according to an embodiment of the disclosure may receive and store the result information on the physical development items of the user.

The processing device 300 may receive a reservation for a user and basic information on the user, and it may transmit the result information on the physical development items of the user to, for example, a personal smart device 400 or a PC (not illustrated) of the user or the guardian of the user. Accordingly, the user or the guardian of the user can easily identify and manage the result information on the physical development items of the user through the personal smart device 400 or the PC.

Meanwhile, the processing device 300 may update the result information on the physical development items obtained from a plurality of users, and it may provide the updated result information on the physical development items as the standard information on the physical development items. That is, because the result information on the physical development items obtained by the plurality of users through the experience device 100 is updated by the processing device 300, and the updated result information on the physical development items is utilized as the standard information on the physical development items, more precise result information on the physical development items of the user who experiences the experience device 100 can be obtained.

With reference to FIG. 1 again, the play experience system 1000 according to an embodiment of the disclosure may further include an immersive media device 500 disposed in each of the plurality of experience rooms 110, 130, and 150 and configured to increase visual, auditory, and tactile sensation for the user who experiences the plurality of experience instruments 120, 140, and 160.

Although FIG. 1 shows that the immersive media device 500 is provided on the outside of each of the plurality of experience rooms 110, 130, and 150, the immersive media device 500 may actually be provided on the inside of each of the plurality of experience rooms 110, 130, and 150. Further, the immersive media device 500 may be provided directly in one or more of the plurality of experience instruments 120, 140, and 160.

In the immersive media device 500, a beam projector, spotlight lighting, and LED lighting may be used as examples to increase the visual sensation for the user; a microphone and a speaker may be used as examples to increase the auditory sensation; and a mist device and a vibration device may be used as examples to increase the tactile sensation. The immersive media device 500 may be provided in the same or different combinations with respect to each of the plurality of experience rooms 110, 130, and 150 or each of the plurality of experience devices 120, 140, and 160.

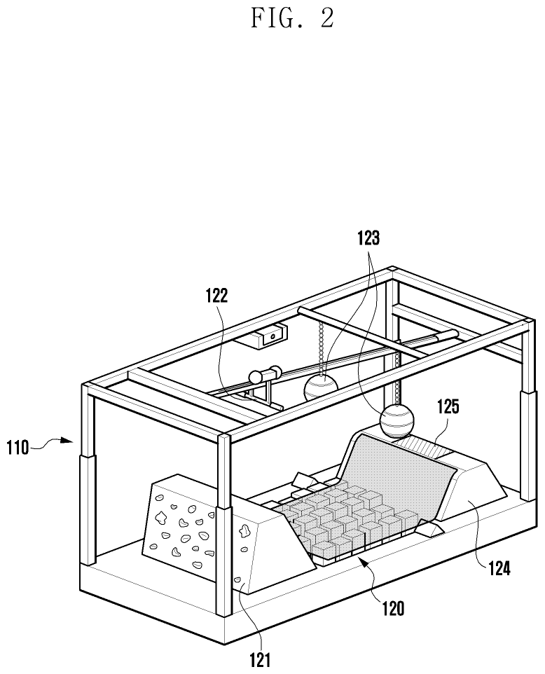

Hereinafter, with reference to FIGS. 2 to 7, several examples of the experience instruments provided in the experience rooms according to an embodiment of the disclosure will be described.

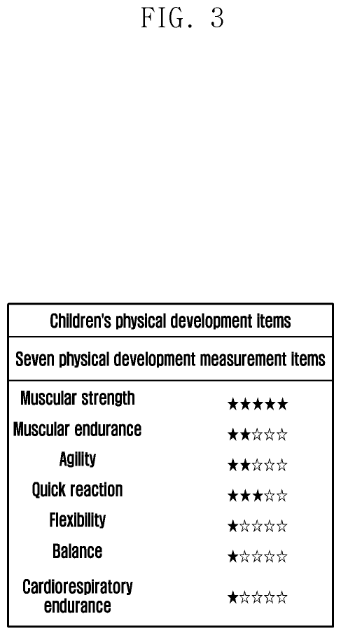

FIG. 2 shows an example of a first experience instrument in which a weight value is given to muscular strength, from among the physical development items; and FIG. 3 illustrates a state in which a star is awarded for physical development items measured by using the first experience instrument of FIG. 2. Here, for convenience of explanation, the plurality of experience rooms 110, 130, and 150 may be called the first to third experience rooms 110, 130, and 150, and the plurality of experience devices 120, 140, and 160 may be called the first to third experience devices 120, 140, and 160.

With reference to FIGS. 2 and 3, the first experience instrument 120 provided in the first experience room 110 is an experience instrument implemented by giving a weight value to muscular strength, from among the physical development items; and it may measure comprehensively the physical development items. Also, it may measure specifically the muscular strength.

The first experience instrument 120 may be configured to include a zip line 122 provided in the space of the first experience room 110, a climbing apparatus 121 provided for climbing to a start location of the zip line 122, and an obstacle 123 provided on a movement line of the zip line 122. Although the illustration shows that two obstacles 123 are provided, it is to be noted that only one obstacle may be provided.

The first experience instrument 120 enables the user to climb up to the start location of the zip line 122 by using a plurality of holders provided on the surface of the climbing apparatus 121, to touch the obstacles 123 with his or her feet during a descent movement along with gripping handles of the zip line 122 from the start location of the zip line 122, and then to land accurately on an arrival location so that the physical development items of the user can be measured comprehensively. Success can be recorded and the success record stored in the processing device 300 only in the case that the user has performed successfully all of the above-described processes (climbing process, descent movement process, obstacle touching process, and landing process) after entering into the first experience instrument 120, whereas failure is recorded and the failure record is not stored in the processing device 300 in the case that even one process is not performed successfully.

Although not illustrated, at the entrance and at the exit of the first experience room 110, a motion recognition sensor configured to sense the entry/exit of a user and a timer configured to check the entry/exit time may be installed, and on the plurality of holders of the climbing apparatus and on the handles of the zip line 122, a pressure sensor configured to sense the grip of the user may be installed. On the obstacles 123, impact sensors configured to sense whether two feet of the user have accurately touched the obstacles 123 (e.g., whether an impact over a predetermined level has been transferred to the obstacles 123) may be installed.

Further, in the first experience room 110, an immersive media device may be installed. Specifically, as the immersive media device, an LED spotlight for notifying entry/success/failure and a speaker for outputting sound may be installed, and a beam projector for outputting a background video for realization while the zip line 122 moves may be installed. Further, a mist spray device spraying mist when the user has arrived at the arrival point may be installed. In this case, success/failure-related background music may be output through the speaker.

A play experience process using the first experience instrument 120 in the first experience room 110 will be described. Whether the user enters into or exits from the first experience room 110 is sensed through the motion recognition sensor, and the time from entry to exit is measured by the timer.

After the entry, the user climbs to the start location of the zip line 122 by gripping the plurality of holders of the climbing apparatus 121. The user moves up to the arrival point of the zip line 122 by gripping the handles of the zip line 122. In this case, the gripping of the holder of the climbing apparatus 121 and the handles of the zip line 122 by the user is sensed by the pressure sensor.

Meanwhile, the impact sensor senses whether the user has applied an impact over the predetermined level to the obstacles 123 by using his or her two feet while moving along the zip line 122. Thereafter, if the user has landed in a landing area 125 of a landing zone 124, whether the user has landed accurately is sensed by the pressure sensor (not illustrated) installed on the landing area 125.

If the experience of the first experience instrument 120 is completed through the above-described processes, the agility and the quick reaction of the user, from among the physical development items, may be measured during the time recorded from entry to exit of the first experience instrument 120; the muscular endurance may be measured from the activity of climbing the climbing apparatus 121 and moving along the zip line 122; and the muscular strength may be measured from the activity of the user kicking the obstacles 123 with his or her feet while moving along the zip line 122.

As illustrated in FIG. 3, in the experience through the comprehensive activity of the first experience instrument 120, a high star rating is given to the muscular strength having the highest weight value, from among the physical development items. Further, because the remaining physical development items (e.g., the flexibility, balance, and cardiorespiratory endurance) excluding the main measurement items are measured comprehensively by the comprehensive activity through the first experience instrument 120, a star may be awarded for the remaining physical development items.

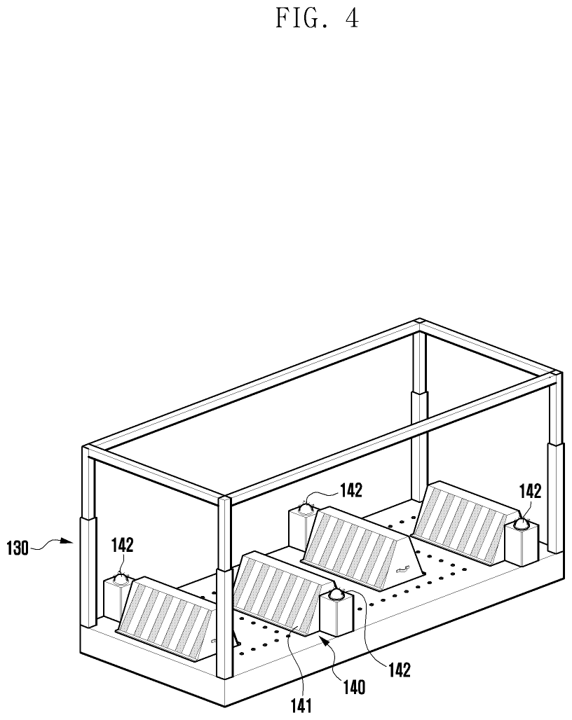

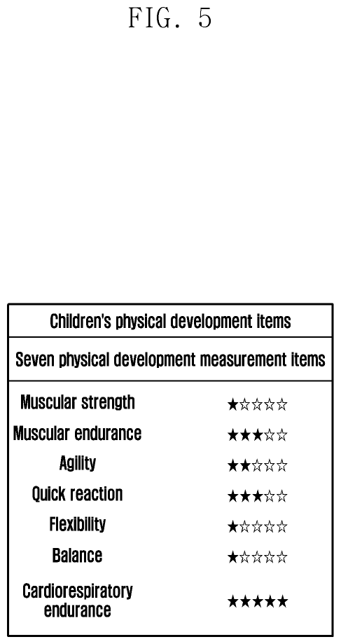

FIG. 4 shows an example of a second experience instrument in which a weight value is given to the cardiorespiratory endurance of the user, from among the physical development items, and FIG. 5 illustrates a state in which a star is awarded for physical development items measured through the second experience instrument of FIG. 4.

With reference to FIGS. 4 and 5, the second experience instrument 140 provided in the second experience room 130 is an experience instrument implemented by giving a weight value to the cardiorespiratory endurance of the user, from among the physical development items; and it may measure comprehensively the physical development items. Also, it may measure specifically the cardiorespiratory endurance of the user.

Although not illustrated, it is preferable that the entrance of the second experience room 130 is disposed to be connected to the exit of the first experience room 110 so that the user having experienced the first experience instrument 120 can experience successively the second experience instrument 140.

The second experience instrument 140 as above may include a plurality of zigzag obstacles 141 provided in the space of the second experience room 130, and on one side of each of the plurality of zigzag obstacles 141, a touch button 142 may be provided.

The second experience instrument 140 enables the user to touch the touch button 142 installed on one side of each of the plurality of zigzag obstacles 141 while moving in a zigzag through the plurality of zigzag obstacles 141; thus, the physical development items of the user can be measured comprehensively. If the second experience instrument 140 is completed within a predetermined time, success can be recorded and the success record stored in the processing device 300, whereas a failure is recorded and the failure record is not stored in the processing device 300 if the time expires before the user touches all touch buttons 142, if the touch buttons 142 are not touched sequentially, or if the user passes over the zigzag obstacles 141.

Although not illustrated, at the entrance and at the exit of the second experience room 130, a motion recognition sensor configured to sense the entry/exit of a user and a timer configured to check the entry/exit time may be installed. In this case, a touch sensor may be installed on each of the touch buttons 142.

Further, in the second experience room 130, an immersive media device may be installed. As the immersive media device, an LED spotlight for notifying entry/success/failure and a speaker for outputting sound may be installed, and an LED for an effect to notify of the remaining time may be installed. In this case, success/failure-related background music may be output through the speaker.

A play experience process using the second experience instrument 140 in the second experience room 130 will be described. Whether the user enters into or exits from the second experience room 130 is sensed through the motion recognition sensor, and the time from entry to exit is measured by the timer.

After the entry into the second experience room 130, if the user touches the touch button 142 installed on one side of each of the plurality of zigzag obstacles 141 while moving in the space between the plurality of zigzag obstacles 141, the touch sensor senses the touch of the user.

As described above, if the experience of the second experience instrument 140 is completed, the agility and the quick reaction of the user, from among the physical development items are measured during the time recorded from entry to exit of the second experience instrument 140, and the cardiorespiratory endurance is measured from the activity of passing through the plurality of zigzag obstacles 141.

As illustrated in FIG. 5, in the experience through the comprehensive activity of the second experience instrument 140, a high star rating is given to the cardiorespiratory endurance having the highest weight value, from among the physical development items. Further, since the remaining physical development items (e.g., the muscular strength, flexibility, and equilibrium) excluding the main measurement items are measured comprehensively by the comprehensive activity through the second experience instrument 140, a star may be awarded for the remaining physical development items.

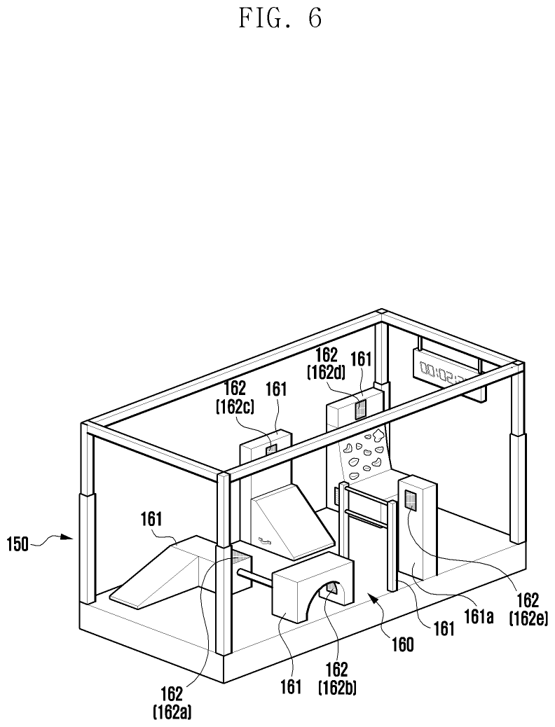

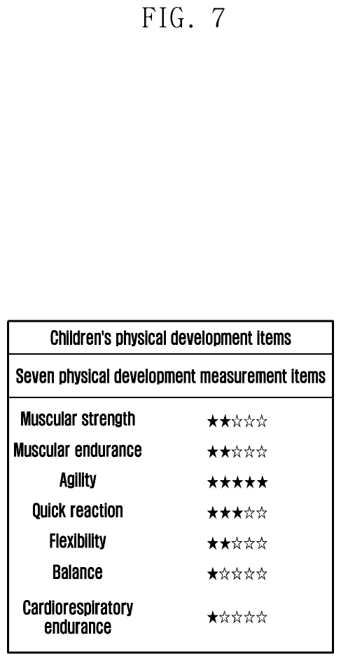

FIG. 6 shows an example of a third experience instrument 160 in which a weight value is given to the agility, from among the physical development items, and FIG. 7 shows a state in which a star is awarded for physical development items measured through the third experience instrument of FIG. 6.

With reference to FIGS. 6 and 7, the third experience instrument 160 provided in the third experience room 150 is an experience instrument implemented by giving the weight value to agility, from among the physical development items; and it may measure comprehensively the physical development items. Also, it may measure specifically the agility.

Although not illustrated, it is preferable that the entrance of the third experience room 150 is disposed to be connected to the exit of the second experience room 130 so that the user having experienced the first experience instrument 120 or the second experience instrument 140 can experience successively the third experience instrument 160.

The third experience instrument 160 as above may include a plurality of fixtures 161 provided in various shapes in the space of the third experience room 150. The plurality of fixtures 161 may include palm pads 162 (162a to 162d) directly provided on blocks constituting the respective fixtures 161, or a palm pad 162 (162a) provided on a separate block 161a. For example, the plurality of fixtures 161 may include a fixture 161 for the user to touch the palm pad 161a on the block, a fixture 161 for the user to touch the palm pad 162b while lying on the floor or bending over, a fixture 161 for the user to touch the palm pad 162c while climbing to the top of a slope, a fixture 161 for the user to touch the palm pad 162d while climbing, and a fixture 162 for the user to touch the palm pad 162 provided on a separate block with another hand or foot while hanging on a bar with one hand or both hands, but the disclosure is not limited thereto. Further, the respective palm pads 162 may be implemented in a lighting method.

The third experience instrument 160 enables the user to measure comprehensively the physical development items of the user by entering the third experience room 150 and by touching the palm pad 162 directly or separately provided to the plurality of fixtures 161. Success may be recorded and the success record stored in the processing device 300 only in the case that the user touches all the palm pads 162 within a time limit, whereas failure is recorded and the failure record is not stored in the processing device 300 in the case that the user fails to touch even one of the palm pads 162.

Although not illustrated, at the entrance and at the exit of the third experience room 150, a motion recognition sensor configured to sense the entry/exit of a user and a timer configured to check the entry/exit time may be installed, and a touch sensor may be installed on each of the palm pads 162.

Further, in the third experience room 150, an immersive media device 500 may be installed. As the immersive media device, an LED spotlight for notifying entry/success/failure and a speaker for outputting sound may be installed, and an LED billboard for counting the time to touch the palm pad 162 may be installed, and a lighting device capable of adjusting brightness in the third experience room 150 may be installed. In this case, success/failure-related background music may be output through the speaker.

A play experience process using the third experience instrument 160 in the third experience room 150 will be described. Whether the user enters into or exits from the third experience room 150 is sensed through the motion recognition sensor, and the time from entry to exit is measured by the timer.

After the entry of the user, the user touches the palm pads 162 provided on the plurality of fixtures 161. If the user touches the palm pad 162, the touch sensor senses the touch.

If the experience of the third experience instrument 160 is completed through the above-described process, the agility and the quick reaction of the user, from among the physical development items, are measured during the time recorded from entry to exit of the third experience instrument 160; and, in particular, the agility is measured from the activity of quickly touching the palm pad 162, and the quick reaction and the muscular endurance are measured from the activity of quickly moving between the plurality of fixtures 161.

As illustrated in FIG. 7, in the experience through the comprehensive activity of the third experience instrument 160, a high star rating is given to the agility having the highest weight value, from among the physical development items. Further, because the remaining physical development items (e.g., the equilibrium and the cardiorespiratory endurance) excluding the main measurement items are measured comprehensively by the comprehensive activity through the third experience instrument 160, a star may be awarded even for the remaining physical development items.

As described above, for example, not only the first experience instrument 120 to the third experience instrument 160 but also more experience instruments in which weight values are given to the respective physical development items may be provided in the plurality of experience rooms 110, 130, and 150 or in additional experience rooms (not illustrated).

Meanwhile, the processing device 300 can obtain the result information based on the measurement information on the physical development items measured comprehensively and obtained through the plurality of experience instruments (the first to third experience instruments 120, 140, and 160 illustrated in FIGS. 2 to 7). Accordingly, the user can obtain more precise result information on the physical development items.

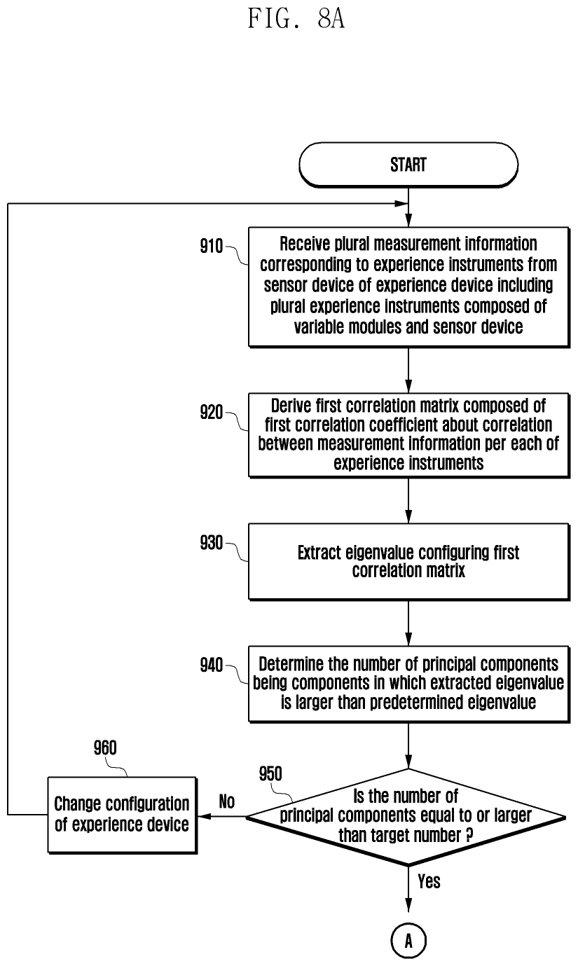

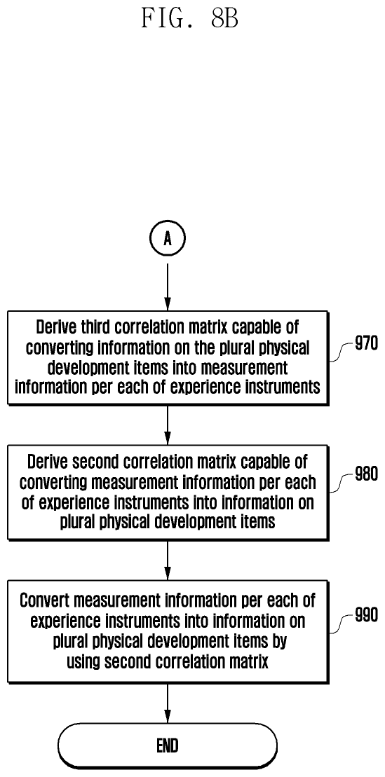

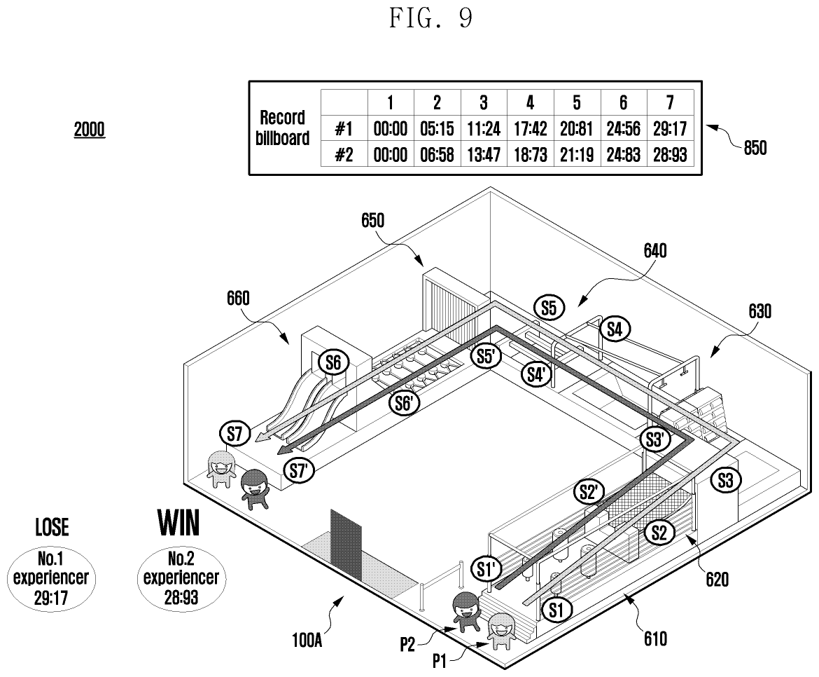

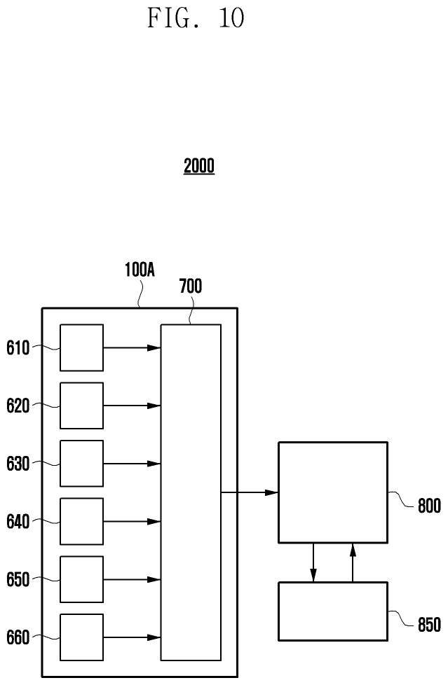

FIGS. 8A and 8B are flowcharts of a physical development status acquisition method using a play experience system according to an embodiment of the disclosure, FIG. 9 is a perspective view of a play experience system used for a physical development status acquisition method using the play experience system according to an embodiment of the disclosure, and FIG. 10 is a configuration diagram schematically illustrating a play experience system used for a physical development status acquisition method using the play experience system according to an embodiment of the disclosure.

A play experience system 2000 illustrated in FIGS. 9 and 10 is a modified example of the play experience system 1000 illustrated in FIG. 1, and in case of FIG. 9, the processing device 800 is not illustrated.

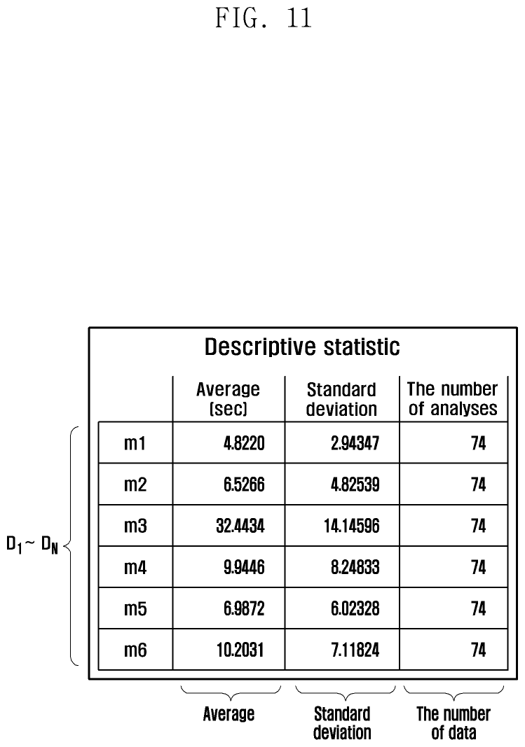

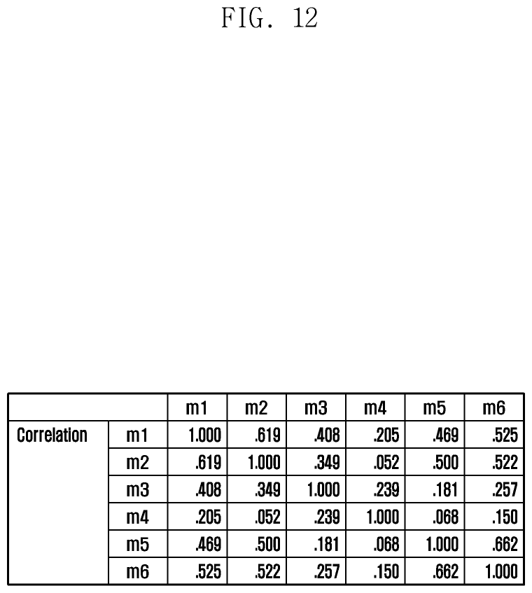

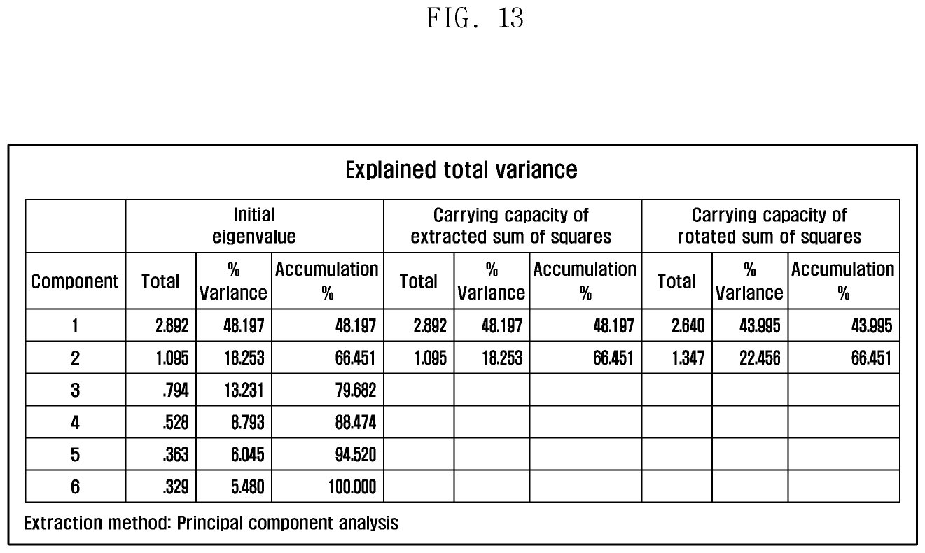

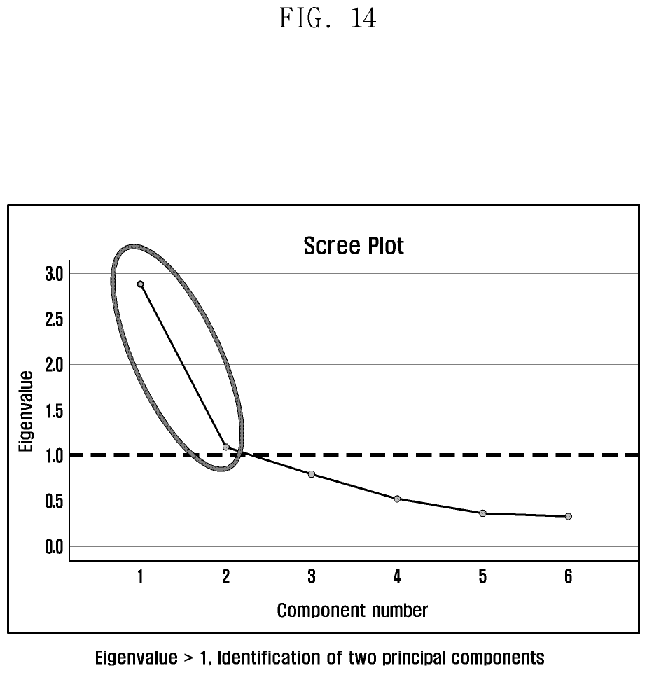

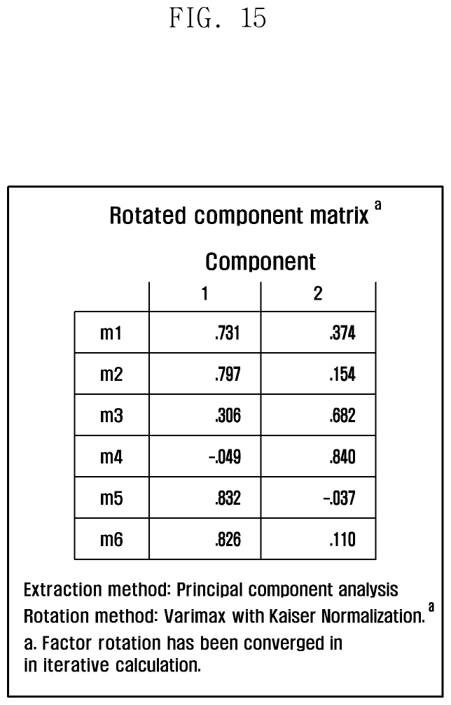

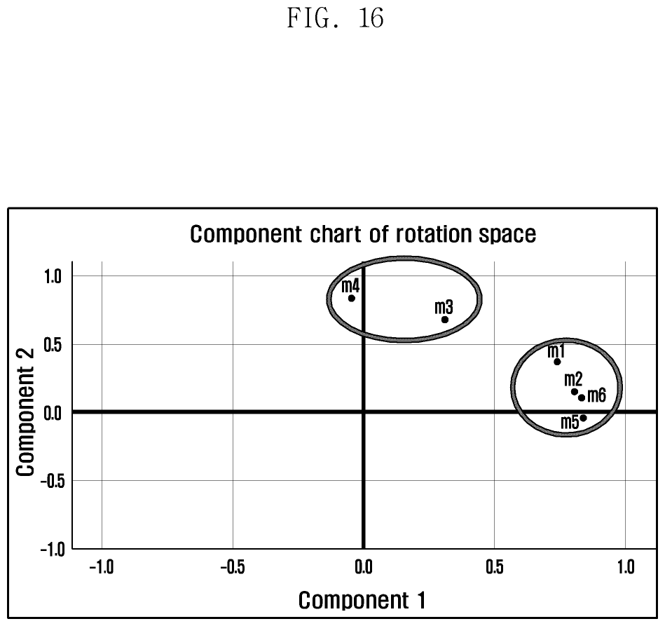

FIG. 11 is a diagram illustrating a table representing an average, standard deviation, and the number of data for each of first to sixth experience instruments of a physical development status acquisition method using a play experience system according to an embodiment of the disclosure, FIG. 12 is a diagram illustrating a first correlation matrix for first to sixth experience instruments of a physical development status acquisition method using a play experience system according to an embodiment of the disclosure, FIG. 13 is a diagram illustrating a table representing eigenvalues by components constituting a first correlation matrix of a physical development status acquisition method using a play experience system according to an embodiment of the disclosure, FIG. 14 is a diagram illustrating eigenvalues by components illustrated in FIG. 13 as a graph, FIG. 15 is a diagram illustrating a table representing correlation values for principal components of first to sixth experience instruments of a physical development status acquisition method using a play experience system according to an embodiment of the disclosure, and FIG. 16 is a diagram illustrating correlation values for principal components of first to sixth experience instruments illustrated in FIG. 15 as a graph.

All values indicated in FIGS. 11 to 16 are values obtained from measurement information described in Tables 1 to 3. Further, m1 to m6 described in FIGS. 11, 12, 15, and 16 mean first to sixth experience instruments 610 to 660.

First, with reference to FIGS. 9 and 10, a play experience system 2000 used for a physical development status acquisition method using the play experience system according to an embodiment of the disclosure will be described as follows.

The play experience system 2000 includes an experience device 100A, a processing device 800, and a display 850.

The experience device 100A includes a plurality experience instruments 610 to 660 composed of variable modules and a sensor device 700. As illustrated in FIGS. 9 and 10, the plurality of experience instruments 610 to 660 may be connected in a course form. Further, the number of experience instruments 610 to 660 of the experience device 100A is 6, and is larger than the number of experience instruments 120, 140, and 160 of the experience device 100 illustrated in FIG. 1 by 3.

The sensor device 700 is a constituent element which senses operations of users P1 and P2 whenever the users P1 and P2 experience the experience instruments 610 to 660, and transmits measurement information per each of experience instruments corresponding to the experience instruments 610 to 660 respectively. For example, as illustrated in FIG. 9, the sensor device 700 may include first to seventh sensors S1 to S7 for the right user P1 and first to seventh sensors S1′ to S7′ for the left user P2. The first to seventh sensors S1 to S7 and S1′ to S7′ may be disposed in order at start points and end points of the first to sixth experience instruments 610 to 660, and may be used to measure the time taken for the users P1 and P2 to pass through the respective experience instruments 610 to 660. Further, the first to seventh sensors S1 to S7 and S1′ to S7′ may be, for example, touch sensors for sensing touches of the users P1 and P2, and may be button-type sensors for sensing pressing of the users P1 and P2.

The processing device 800 may receive the measurement information per each of experience instruments corresponding to the experience instruments 610 to 660 respectively from the sensor device 700, and may derive a first correlation matrix composed of first correlation coefficients for the correlation between the measurement information by the experience instruments. Further, the processing device 800 may determine the number of principal components being components in which an eigenvalue constituting the first correlation matrix is larger than a predetermined eigenvalue, and may derive a second correlation matrix capable of converting the measurement information per each of the experience instruments into information on a plurality of physical development items in accordance with the number of the principal components. Further, the processing device 800 may convert the measurement information per each of the experience instruments into the information on the plurality of physical development items by using the second correlation matrix.

The display 850 is a constituent element displaying a start signal to the users P1 and P2 or displaying the time when the users pass through the respective experience instruments 610 to 660, and may be composed of a touchscreen panel display or a display having no touch function.

Next, with reference to FIGS. 8A to 16, the physical development status acquisition method using the play experience system according to an embodiment of the disclosure will be described.

At step 910, the processing device 800 receives the measurement information per each of the experience instruments corresponding to the experience instruments 610 to 660 respectively from the sensor device 700 of the experience device 100A including the plurality of experience instruments 610 to 660 composed of the variable modules and the sensor device 700. For example, the measurement information per each of the experience instruments may be information on the time taken to pass through the respective experience instruments (first EI˜ sixth EI) 610 to 660 as described in the following Tables 1 to 3.

TABLE 1

| | | | Measurement information (unit: sec) | | | | | |
|---|---|---|---|---|---|---|---|---|
| user | age | gender | first EI | second EI | third EI | fourth EI | fifth EI | sixth EI |
| 1 | 12 | 1 | 1.11 | 1.11 | 1.21 | 1.01 | 1.11 | 1.11 |
| 2 | 11 | 1 | 1.41 | 0.81 | 34.84 | 11.82 | 0.2 | 0.2 |
| 3 | 12 | 1 | 3.53 | 0.81 | 49.79 | 17.58 | 2.42 | 6.56 |
| 4 | 11 | 1 | 1.61 | 0.2 | 33.43 | 20.31 | 1.21 | 1.41 |
| 5 | 11 | 1 | 1.71 | 0.41 | 53.43 | 29.08 | 0.81 | 1.01 |
| 6 | 12 | 1 | 7.87 | 0.61 | 32.72 | 29.09 | 0.81 | 1.01 |
| 7 | 11 | 1 | 1.41 | 1.62 | 34.03 | 12.22 | 1.42 | 1.21 |
| 8 | 11 | 1 | 1.61 | 4.24 | 42.83 | 8.78 | 0.31 | 0.91 |
| 9 | 12 | 1 | 1.51 | 0.4 | 34.04 | 5.15 | 1.21 | 1.22 |
| 10 | 12 | 1 | 3.23 | 1.71 | 32.43 | 17.77 | 0.51 | 0.91 |
| 11 | 11 | 1 | 1.31 | 1.72 | 37.47 | 5.65 | 1.62 | 0.6 |
| 12 | 11 | 1 | 0.91 | 0.91 | 0.91 | 0.91 | 0.91 | 0.91 |
| 13 | 12 | 1 | 1.11 | 1.11 | 1.21 | 1.01 | 1.11 | 1.11 |
| 14 | 12 | 1 | 1.11 | 1.11 | 1.21 | 1.01 | 1.11 | 1.11 |
| 15 | 11 | 1 | 1.01 | 1.01 | 1.01 | 1.01 | 1.01 | 1.01 |
| 16 | 11 | 1 | 1.11 | 1.11 | 1.21 | 1.01 | 1.11 | 1.11 |
| 17 | 12 | 1 | 1.41 | 2.02 | 38.99 | 12.52 | 0.5 | 1.12 |
| 18 | 11 | 1 | 1.91 | 2.13 | 36.05 | 13.03 | 1.01 | 1.31 |
| 19 | 11 | 1 | 4.94 | 5.26 | 41 | 7.17 | 6.77 | 11.82 |
| 20 | 11 | 1 | 3.23 | 7.77 | 43.64 | 9.39 | 6.36 | 5.36 |
| 21 | 11 | 1 | 11.81 | 5.15 | 44.04 | 8.99 | 4.34 | 20.91 |
| 22 | 11 | 1 | 6.86 | 11.52 | 48.98 | 3.44 | 5.75 | 16.97 |
| 23 | 12 | 1 | 7.57 | 11.21 | 38.38 | 7.17 | 12.63 | 11.31 |
| 24 | 11 | 1 | 6.96 | 2.63 | 41.41 | 5.35 | 8.39 | 11.81 |
| 25 | 12 | 1 | 2.92 | 4.75 | 28.69 | 4.54 | 7.58 | 13.23 |
| 26 | 12 | 1 | 11 | 3.34 | 20.4 | 16.06 | 4.85 | 4.54 |

TABLE 2

| user | age | gender | first EI | second EI | third EI | fourth EI | fifth EI | sixth EI |
|---|---|---|---|---|---|---|---|---|
| 27 | 11 | 1 | 2.82 | 3.24 | 42.62 | 3.33 | 3.64 | 4.74 |
| 28 | 11 | 1 | 2.52 | 6.67 | 39.79 | 9.6 | 3.93 | 7.58 |
| 29 | 12 | 1 | 3.03 | 3.73 | 27.47 | 4.15 | 10.8 | 20.1 |
| 30 | 11 | 1 | 2.62 | 3.94 | 37.88 | 4.34 | 4.95 | 11.41 |
| 31 | 12 | 1 | 4.52 | 2.26 | 24.37 | 4.28 | 8.8 | 8.4 |
| 32 | 11 | 1 | 5.95 | 5.11 | 33.61 | 10.01 | 4.31 | 7.16 |
| 33 | 11 | 1 | 5.73 | 3.07 | 33.79 | 28.46 | 0.83 | 13.71 |
| 34 | 11 | 1 | 6.54 | 10.66 | 27.25 | 18.23 | 15.13 | 13.33 |
| 35 | 11 | 1 | 4.71 | 3.28 | 34.01 | 28.46 | 12.49 | 27.03 |
| 36 | 12 | 1 | 4.3 | 8.99 | 27.66 | 36.45 | 11.07 | 27.65 |
| 37 | 10 | 2 | 5.51 | 2.89 | 52.63 | 11.26 | 13.3 | 17.2 |
| 38 | 10 | 2 | 7.06 | 9.12 | 37.48 | 4.49 | 5.33 | 6.36 |
| 39 | 5 | 2 | 8.8 | 13.92 | 60.01 | 11.66 | 3.5 | 12.06 |
| 40 | 5 | 2 | 5.73 | 8.8 | 54.87 | 12.9 | 3.91 | 8.8 |
| 41 | 12 | 1 | 3.87 | 7.16 | 26.85 | 5.33 | 30.5 | 12.9 |
| 42 | 11 | 1 | 4.09 | 9.2 | 27.87 | 3.91 | 5.11 | 10.23 |
| 43 | 11 | 1 | 4.03 | 8.18 | 25.92 | 4.18 | 4.93 | 8.59 |
| 44 | 12 | 1 | 3.68 | 8.75 | 25.26 | 5.3 | 5.77 | 7.56 |
| 45 | 12 | 1 | 3.87 | 7.6 | 15.34 | 5.15 | 4.89 | 10.85 |
| 46 | 10 | 2 | 3.47 | 5.95 | 22.94 | 4.49 | 4.72 | 8.4 |
| 47 | 8 | 1 | 3.44 | 6.2 | 24.39 | 4.1 | 5.95 | 7.38 |
| 48 | 10 | 2 | 3.37 | 5.24 | 25.61 | 3.25 | 5.34 | 7.78 |
| 49 | 10 | 2 | 3.19 | 5.42 | 26.73 | 3 | 3.57 | 7.56 |
| 50 | 8 | 1 | 3.87 | 5.12 | 10.44 | 4.53 | 4.09 | 10.23 |
| 51 | 11 | 1 | 9.42 | 10.85 | 26.63 | 21.3 | 6.54 | 13.73 |
| 52 | 11 | 1 | 4.71 | 10.85 | 27.46 | 22.32 | 8.4 | 8.19 |
| 53 | 11 | 1 | 5.33 | 15.56 | 44.86 | 8.99 | 10.66 | 15.16 |

TABLE 3

| user | age | gender | first EI | second EI | third EI | fourth EI | fifth EI | sixth EI |
|---|---|---|---|---|---|---|---|---|
| 54 | 11 | 1 | 6.75 | 3.48 | 38.71 | 8.19 | 5.33 | 9.83 |
| 55 | 11 | 1 | 11.06 | 9.02 | 51.22 | 5.3 | 20.08 | 19.26 |
| 56 | 12 | 1 | 14.32 | 15.99 | 55.31 | 6.14 | 17.6 | 15.57 |
| 57 | 11 | 1 | 3.68 | 6.17 | 27.44 | 6.35 | 5.74 | 10.01 |
| 58 | 12 | 1 | 9.2 | 9.64 | 29.48 | 4.93 | 7.57 | 18.01 |
| 59 | 11 | 1 | 4.49 | 9.02 | 18.63 | 7.6 | 19.65 | 19.56 |
| 60 | 12 | 1 | 3.22 | 4.09 | 31.56 | 3.69 | 20.06 | 24.18 |
| 61 | 11 | 1 | 3.9 | 5.12 | 24.36 | 7.6 | 5.73 | 23.13 |
| 62 | 11 | 1 | 5.73 | 11.72 | 33.14 | 7.87 | 10.36 | 23.43 |
| 63 | 12 | 1 | 7.37 | 11.66 | 34.41 | 6.54 | 10.51 | 23.28 |
| 64 | 12 | 1 | 5.82 | 9.33 | 23.56 | 9.21 | 6.73 | 8.83 |
| 65 | 11 | 1 | 6.1 | 8.25 | 28.02 | 5.52 | 5.92 | 9.08 |
| 66 | 12 | 1 | 8.02 | 6.73 | 25.2 | 25.58 | 23.16 | 12.49 |
| 67 | 13 | 1 | 5.51 | 8.19 | 67.21 | 7.96 | 8.4 | 13.74 |
| 68 | 13 | 1 | 10.63 | 20.27 | 55.31 | 5.92 | 15.99 | 17.61 |
| 69 | 6 | 1 | 9.85 | 20.28 | 37.26 | 31.74 | 15.57 | 17.6 |
| 70 | 12 | 1 | 6.13 | 10.67 | 25.17 | 18.85 | 6.97 | 12.28 |
| 71 | 12 | 1 | 6.97 | 13.3 | 35.43 | 4.53 | 8.59 | 11.68 |
| 72 | 12 | 1 | 4.71 | 10.44 | 31.97 | 3.87 | 9.83 | 11.9 |
| 73 | 12 | 1 | 4.3 | 18.45 | 45.26 | 9.83 | 6.75 | 10.05 |
| 74 | 8 | 2 | 2.72 | 4.72 | 45.07 | 14.14 | 8.99 | 9.64 |

At step 920, the processing device 800 derives the first correlation matrix composed of the first correlation coefficients about the correlation between the measurement information per each of the measurement instruments. For example, the first correlation coefficients of the first correlation matrix may be obtained using a covariate between the measurement information per each of the experience instruments and the standard deviation of the measurement information per each of the experience instruments.

In Mathematical expression 1, A denotes the first correlation matrix. As shown in Mathematical expression 1, it can be known that the first correlation coefficient about the correlation between the measurement information for the same experience instrument is 1.

In Mathematical expression 2, a_{xy} is the first correlation coefficient about the correlation between the measurement information on the x-th experience instrument and the measurement information on the y-th experience instrument, Sₓ is the standard deviation of the measurement information on the x-th experience instrument, and S_{y} is the standard deviation of the measurement information on the y-th experience instrument. In Mathematical expressions 2 and 3, COV_{xy} is a covariate between the measurement information on the x-th experience instrument and the measurement information on the y-th experience instrument. In Mathematical expression 3, Tₓ is the measurement information on the x-th experience instrument, Aₓ is an average value of the measurement information on the x-th experience instrument, T_{y} is the measurement information by the users for the y-th experience instrument, A_{y} is an average value of the measurement information on the y-th experience instrument, and Z is the number of users.

$$A = ( \begin{matrix}1 & {a}_{12} & … & {a}_{1 N} \\ {a}_{21} & 1 & … & {a}_{2 N} \\ ⋮ & ⋮ & ⋱ & ⋮ \\ {a}_{N 1} & {a}_{N 2} & … & 1\end{matrix} ) \qquad [ Mathematical expression 1 ]$$

$${a}_{x y} = \frac{{COV}_{x y}}{{S}_{x} \times {S}_{y}} \qquad [ Mathematical expression 2 ]$$

$${COV}_{x y} = \frac{\sum [ ( {T}_{x} - {A}_{x} ) \times ( {T}_{y} - {A}_{x} ) ]}{Z - 1} \qquad [ Mathematical expression 3 ]$$

Further, the average value and the standard deviation of the measurement information per each of the experience instruments for the first to sixth experience instruments (first EI˜sixth EI) 610 to 660 obtained using the measurement information per each of the experience instruments described in Tables 1 to 3 and the first correlation matrix are shown in FIGS. 11 and 12, respectively. The average value, the standard deviation, and the first correlation matrix as described above may be obtained using the known statistical package for the social sciences (SPSS) program.

Further, the step 920 may include data normalization which makes the influence on the data by the items equal to one another by converting the average of the measurement information per each of the experience instruments into 0 and converting the standard deviation of the measurement information per each of the experience instruments into 1. For example, the data normalization may be automatically performed using the covariate COV_{xy} and the standard deviation Sₓ and S_{y} of the Mathematical expressions 2 and 3.

At step 930, the processing device 800 extracts eigenvalues constituting the first correlation matrix. For example, the eigenvalue extraction may be obtained through principle component analysis (PCA) using the above-described known SPSS program. The eigenvalues by components constituting the first correlation matrix illustrated in FIG. 12 are shown in FIG. 13. Specifically, as illustrated in FIG. 13, it has been identified that the eigenvalue of the first component is 2.892, the eigenvalue of the second component is 1.09, the eigenvalue of the third component is 0.794, the eigenvalue of the fourth component is 0.528, the eigenvalue of the fifth component is 0.363, and the eigenvalue of the sixth component is 0.329.

At step 940, the processing apparatus 800 determines the number of principal components that is the component in which the extracted eigenvalue is larger than a predetermined eigenvalue. The predetermined eigenvalue may be, for example, “1”, and as illustrated in FIG. 14, by determining the first component and the second component having the eigenvalue that is larger than the predetermined eigenvalue of “1” as the principal components, it can be determined that the number of the principal components is 2.

At step 950, the processing device 800 may determine whether the number of principal components is equal to or larger than a target number. The “target number” may be the number of physical development items intended to be obtained from the experience device 100A.

If it is determined that the number of principal components is smaller than the target number at step 950, a manager (not illustrated) managing the play experience system 2000 may change the configuration of the experience device 100A at step 960. Such change of the configuration of the experience device 100A may include one or more of the change of the configuration of the experience instruments 610 to 660 and the change of the configuration of the sensor device 700.

For example, the change of the configuration of the experience instruments 610 to 660 may be addition of new experience instruments and/or the change of the configuration of one or more of the existing experience instruments 610 to 660. The change of the configuration of the sensor device 700 may be addition of a new sensor device capable of generating different kinds of measurement information excluding the measurement information per each of the existing experience instruments and/or the change of the configuration of one or more of sensors S1 to S7 and S1′ to S7′ of the existing sensor device 700. If it is assumed that the measurement information per each of the existing experience instruments is information on the time taken to pass through the respective experience instruments 610 to 660, the measurement information that may be generated by the new sensor device may be, for example, information on the number of touches of the sensor while the users P1 and P2 experience one experience instrument, or information on the reaction time when the users P1 and P2 react on signals generated from the play experience system 2000.

At step 950, if it is determined that the number of principal components is smaller than the target number, although not illustrated in FIGS. 8A and 8B, a step of determining the number of principal components may be performed through combination of the measurement information per each of the existing experience instruments and the measurement information per each of the additional experience instruments. The step of determining the number of principal components through the combination of the measurement information per each of the existing experience instruments and the measurement information per each of the additional experience instruments may be performed by the same method as that at step 910 to 940 as described above. In relation to this, the measurement information per each of the existing experience instruments may include error data, such as “0.2 second” being the measurement information of the fifth and sixth experience instruments 650 and 660 of the second user described in Table 1. The error data may be generated in case that the user does not properly perform the experience instrument and/or due to mechanical malfunction, and may cause the number of principal components capable of being extracted from the experience device 100A to be reduced. Accordingly, by increasing the number of measurement information per each of additional experience instruments, the number of the principal components can be increased through reduction of the error data rate.

If the configuration of the experience device 100A is changed at step 960, the processing device 800, at step 910, re-receives the measurement information per each of the experience instruments from the sensor device 700 of the experience device 100A.

If it is determined that the number of principal components is equal to or larger than the target number at step 950, the processing device 800, at step 970, derives the third correlation matrix capable of converting information on a plurality of physical development items into the measurement information per each of the experience instruments. The third correlation matrix may be composed of the third correlation coefficients about the correlation between the measurement information per each of the experience instruments and information on pre-stored physical development items, and for example, may be described as in the following Mathematical expressions 4 and 5.

In Mathematical expression 4, c₁₁ to c_{NM} are the third correlation coefficients about the correlation between the measurement information per each of the experience instruments and the information on the pre-stored physical development items, and for example, may be obtained using the covariate and the standard deviation of the measurement information per each of the experience instruments and the information on the pre-stored physical development items.

In Mathematical expressions 4 and 5, C denotes the third correlation coefficient, and in Mathematical expression 5, D₁ to D_{N} denote the measurement information per each of the experience instruments, and X₁ to X_{M} denote information on pre-stored physical development items.

$$C = ( \begin{matrix}{c}_{11} & {c}_{12} & … & {c}_{1 M} \\ {c}_{21} & {c}_{22} & … & {c}_{2 M} \\ ⋮ & ⋮ & ⋱ & ⋮ \\ {c}_{N 1} & {c}_{N 2} & ⋯ & {c}_{NM}\end{matrix} ) \qquad [ Mathematical expression 4 ]$$

$$( \begin{matrix}\begin{matrix}\begin{matrix}{D}_{1} \\ {D}_{2}\end{matrix} \\ ⋮\end{matrix} \\ {D}_{N}\end{matrix} ) = C \times ( \begin{matrix}\begin{matrix}\begin{matrix}{X}_{1} \\ {X}_{2}\end{matrix} \\ ⋮\end{matrix} \\ {X}_{M}\end{matrix} ) \qquad [ Mathematical expression 5 ]$$

At step 980, the processing device 800 derives the second correlation matrix capable of converting the measurement information per each of the experience instruments into the information on the plurality of physical development items. For example, the second correlation matrix may be obtained by an inverse matrix C⁻¹ of the third correlation matrix C as disclosed in the following Mathematical expressions 6 and 7.

In Mathematical expression 6, b₁₁ to b_{MN} are the second correlation coefficients about the correlation between the information on the physical development items and the measurement information per each of the experience instruments, and in Mathematical expressions 6 and 7, D₁ to D_{N} denote the measurement information per each of the experience instruments, and X₁ to X_{M} denote the information on the physical development items.

$$B = {C}^{- 1} = ( \begin{matrix}{b}_{11} & {b}_{12} & … & {b}_{1 N} \\ {b}_{21} & {b}_{22} & … & {b}_{2 N} \\ ⋮ & ⋮ & ⋱ & ⋮ \\ {b}_{M 1} & {b}_{M 2} & … & {b}_{MN}\end{matrix} ) \qquad [ Mathematical expression 6 ]$$

$$B \times ( \begin{matrix}\begin{matrix}\begin{matrix}{D}_{1} \\ {D}_{2}\end{matrix} \\ ⋮\end{matrix} \\ {D}_{N}\end{matrix} ) = ( \begin{matrix}\begin{matrix}\begin{matrix}{X}_{1} \\ {X}_{2}\end{matrix} \\ ⋮\end{matrix} \\ {X}_{M}\end{matrix} ) \qquad [ Mathematical expression 7 ]$$

Further, in the present embodiment, although the second correlation matrix is indirectly derived by the inverse matrix of the third correlation matrix, the derivation of the second correlation matrix is not limited thereto, and the second correlation matrix may be directly derived by obtaining the second correlation coefficients by using the information on the pre-stored physical development items and the measurement information per each of the experience instruments.

Further, deriving the second correlation matrix or deriving the third correlation matrix, which is performed if it is determined that the number of principal components is equal to or larger than the target number at operation 950, may include determining the kind of the physical development item that can be obtained from the experience device 100A by calculating correlation values of the experience instruments 610 to 660 for the respective principal components.

With reference to FIGS. 15 and 16, determination of the kind of the physical development item that can be obtained from the experience device 100A may be performed, for example, through a rotated component matrix by Varimax with Kaiser Normalization using the SPSS program. As illustrated in FIGS. 15 and 16, it has been calculated that the third and fourth experience instruments 630 and 640 have high correlation values for the first principal component, and the first, second, fifth, and sixth experience instruments 610, 620, 650, and 660 have high correlation values for the second principal component. In relation to this, in case that the expected physical development items by the experience instruments as in Table 4 below are pre-stored in the processing device 800, the processing device 800 may determine “quick reaction” being the common expected physical development item of the third and fourth experience instruments 630 and 640 and “agility” being the common expected physical development item of the first, second, fifth, and sixth experience instruments 610, 620, 650, and 660 as the kinds of the physical development items capable of being obtained from the experience device 100A.

TABLE 4

| | Expected physical development |
|---|---|
| Experience instrument | items |
| First experience | Quick reaction, Agility |
| instrument | |
| Second experience | Muscular strength, Agility |
| instrument | |
| Third experience | Muscular strength, Quick |
| instrument | reaction |
| Fourth experience | Quick reaction, Balance |
| instrument | |
| Fifth experience | Agility, Balance |
| instrument | |
| Sixth experience | Quick reaction, Agility |
| instrument | |

At step 990, the processing device 800 converts the measurement information per each of the experience instruments into the information on the plurality of physical development items by using the second correlation matrix.

According to the physical development status acquisition method using the play experience system according to the present embodiment, since the number of principal components is determined in accordance with the eigenvalues constituting the first correlation matrix composed of the first correlation coefficients about the correlation between the measurement information per each of the experience instruments, and the second correlation matrix capable of converting the measurement information per each of the experience instruments into the information on the plurality of physical development items in accordance with the number of the principal components is derived, all pieces of desired information on the plurality of physical development items can be accurately acquired through accurate analysis of the correlation between the measurement information obtained from the experience through the experience instruments 610 to 660 and the plurality of physical development items.

### INDUSTRIAL APPLICABILITY

Although embodiments of the disclosure have been described as above, the scope of the disclosure is not necessarily limited thereto, and any modifications and variations can be embodied within the category of the technical idea of the disclosure.

1. A play experience system comprising:
an experience device including a plurality of experience rooms configured to provide spaces in which a user can carry out a play-type experience and composed of variable modules that are connected to form a course, and a plurality of experience instruments provided in each of the plurality of experience rooms for the user to experience the play-type experience so that physical development items of the user are measured comprehensively;
a sensor device including at least one or more sensors being disposed in a predetermined area of each of the plurality of experience instruments and configured to measure comprehensively the physical development items while the user experiences the plurality of experience instruments; and
a processing device configured to receive body composition information and heart rate information of the user and comprehensive measurement information on the physical development items of the user from the at least one or more sensors, and to acquire result information on a current level of the physical development items of the user by comparing the received comprehensive measurement information on the physical development items with pre-stored standard information on the physical development items.

2. A physical development status acquisition method using a play experience system, the method comprising steps of:
receiving, from a sensor device wherein an experience device includes a plurality of experience instruments composed of variable modules and the sensor device, measurement information per each of the experience instruments corresponding to the experience instruments, respectively;
deriving a first correlation matrix composed of first correlation coefficients about a correlation among the measurement information per each of the experience instruments;
determining a number of a principal component in which each eigenvalue of components constituting the first correlation matrix is larger than a predetermined eigenvalue;
deriving a second correlation matrix capable of converting the measurement information per each of the experience instruments into information on a plurality of physical development items in accordance with the number of the principal component; and
converting the measurement information per each of the experience instruments into the information on the plurality of physical development items by using the second correlation matrix.

3. The physical development status acquisition method of claim 2, wherein the step of deriving the second correlation matrix comprises a step of changing a configuration of the experience device in accordance with the number of the principal component.

4. The physical development status acquisition method of claim 2, wherein the step of deriving the second correlation matrix comprises a step of determining the number of the principal component by a combination of the measurement information per each of the existing experience instruments and measurement information per each of additional experience instruments in accordance with the number of the principal component.

5. The physical development status acquisition method of claim 2, wherein the step of deriving the second correlation matrix comprises steps of:
deriving a third correlation matrix capable of converting the information on the plurality of physical development items into the measurement information per each of the experience instruments; and
deriving the second correlation matrix by an inverse matrix of the third correlation matrix.

6. The physical development status acquisition method of claim 2, wherein the first correlation coefficients are obtained by using a covariate between the measurement information per each of the experience instruments and a standard deviation of the measurement information per each of the experience instruments.

7. The physical development status acquisition method of claim 2, wherein the step of deriving the second correlation matrix derives the second correlation matrix depending on whether the number of the principal component is equal to or larger than a target number of the physical development items intended to obtain from the experience device.

8. The physical development status acquisition method of claim 2, wherein the step of deriving the second correlation matrix comprises a step of determining a type of the physical development items capable of being obtained from the experience device by calculating a correlation value of the experience instruments for the principal component.

9. The physical development status acquisition method of claim 3, wherein the step of changing the configuration of the experience device comprises one or more of changing a configuration of the experience instruments and changing a configuration of the sensor device.
